(19) United States
(12) Patent Application Publication
(10) Pub. No.: US 2016/0175646 A1
(43) Pub. Date: Jun. 23, 2016

(54) METHOD AND SYSTEM FOR IMPROVING BIOMECHANICS WITH IMMEDIATE PRESCRIPTIVE FEEDBACK

(71) Applicant: Vibrado Technologies, Inc., Sunnyvale, CA (US)

(72) Inventors: Quinn A. Jacobson, Sunnyvale, CA (US); Cynthia Kuo, Mountain View, CA (US)

(21) Appl. No.: 14/702,304

(22) Filed: May 1, 2015

Related U.S. Application Data
(60) Provisional application No. 62/093,186, filed on Dec. 17, 2014.

(51) Int. Cl. A63B 24/00; A61B 5/00; A61B 5/11

(52) U.S. Cl. CPC A63B 24/0003; A61B 5/11; A61B 5/486; A63B 24/0062

(57) ABSTRACT

Prescriptive feedback about an activity performed by a person can be generated by a system having on-body sensors attached to the person's body. Data from the on-body sensors is analyzed by a processor device which instructs feedback devices to generate indicators that guide the person to perform the activity closer to a model of the activity. The indicators can be visual, audible, or haptic, and they can be generated on the person's body in real time.

### CROSS-REFERENCE TO RELATED APPLICATION

This application claims the benefit of U.S. Provisional Application No. 62/093,186, filed Dec. 17, 2014, which is incorporated herein by reference in its entirety and for all purposes.

### FIELD

The invention relates, in general to, equipment for evaluating biomechanical motion, and more particularly, to a method and system for providing prescriptive feedback for improving biomechanics.

### BACKGROUND

Learning or perfecting a skill requiring physical dexterity is challenging. But there are many situations where learning or perfecting a skill can drastically increase the quality of one's life. Examples include everything from a child learning to play a sport like basketball or an adult perfecting a hobby like golf to applications like regaining strength and mobility after knee surgery or learning to feed oneself after a stroke or cerebrovascular accident.

Traditionally learning and mastering a skill requiring physical dexterity consists of both working with an expert and practicing on one's own. The expert can explain what to do, demonstrate how to do it, and guide the individual to perform the activity correctly.

The act of explaining and demonstrating a skill can be performed in person or through videos or other means. Anyone can watch a video on how to shoot a basketball free throw, hit a golf ball, or perform post-surgery exercises. Unfortunately, without the guidance of an expert, a beginner is likely to perform a skill with incorrect form. Learning and reinforcing muscle memory of the wrong form often limits an individual's performance and benefit. For an athlete this limits their ability to improve and become more competitive. For someone in physical therapy or rehabilitation, performing the exercises incorrectly can not only limit the benefit but can also risk further injury.

For elite athletes, the problem shifts to the need to achieve consistency in performance. All advanced athletes can perform the skills needed to win some of the time, but champions perform the skills correctly all of the time. Dedicated athletes practice to reinforce the muscle memory for consistent performance.

A key to success in learning or perfecting a skill is to have an expert visually observe you perform the skill and then guide with immediate feedback. When done successfully the expert, identifies what motion is being performed incorrectly and provides prescriptive feedback on what to do differently to achieve the best result. The more detail the expert can observe, as well as the more the expert understands the biomechanics, the better the expert can identify what changes should be prescribed. Also, the better the expert can convey the prescribed changes to the individual, the more efficiently the individual can learn and perfect the skill.

There are a number of trade-offs an expert must consider when observing and coaching an individual. The human eye is often not sufficient to observe in detail the biomechanics of an individual. This may be caused by the individual moving too fast for the expert to accurately observe the motion, or it may due to aspects of biomechanics, such as which muscle group is used to initiate or perform a motion, that is inherently difficult to observe with the naked eye. High-speed video capture and slow-motion replay can help, but these conventional methods delay the time from when the individual performs the activity and when the feedback is provided, which slows down the learning process and limits the ability for the individual to feel what was done incorrectly. Similarly, attaching sensors such as myography sensors to detect muscle activity and having the expert interpret the resulting sensor information slows down the feedback process.

Ultimately most individuals cannot have an expert available at all times to help them learn and perfect skills. The individual spends most of the time practicing without an expert. Without an expert present, the individual tries to reinforce correct biomechanics by a combination of remembering the correct form, sensing whether the activity was performed with correct form, and/or observing the result of the activity. All three of these are problematic. People often forget the correct form when an expert is not present to provide guidance. If one practices with incorrect form, one is at least wasting practice time and likely reinforcing bad habits, and in the worst case putting oneself at increased risk of injury. For most people, personal awareness of biomechanics is limited. Most people cannot sense personal body position or biomechanics accurately enough to perceive the subtle differences between correct and incorrect form. Also, observing the results of activity (e.g., whether a basketball entered the hoop or whether a baseball travelled the correct speed and trajectory) is often not sufficient to determine whether the activity was performed with correct form. One can lift a weight, hit a ball or perform whatever activity with some success even with very poor form, which can then reinforce bad biomechanical habits if no expert is present to point out deficiencies in form.

On-body technology offers an opportunity to revolutionize how people learn and perfect skills requiring physical dexterity. Unfortunately most of today's wearable technology systems fall short of this potential. Conventional systems do not observe the biomechanics of an individual but instead observe the result. These systems focus on observing extrinsic events (e.g., how hard they hit the ball, how high they throw the ball, and how many strides they take) rather than focusing on how the activity was performed. Also, conventional systems provide data to the individual but do not interpret the data and do not provide actionable, prescriptive feedback.

What is needed is system and method that makes use of wearable sensors to monitor an individual's biomechanics in real-time to help (1) an expert analyze the individual's biomechanics more accurately, and (2) allow individuals to have their practice interpreted and feedback provided when no expert is present. With no expert present, the system and method can analyze the biomechanical data from the sensors to provide prescriptive feedback on what to do differently to achieve more optimal motion. What is also needed a means for providing prescriptive feedback that efficiently communicates to an individual how to modify his or her biomechanics.

### SUMMARY

Briefly and in general terms, the present invention is directed to a method, system, and computer readable medium for generating prescriptive feedback about an activity performed by a person.

In aspects of the present invention, a method comprises receiving data from on-body sensors attached to the body of a person performing an activity that includes biomechanical motions, analyzing the data received from the on-body sensors, and providing prescriptive feedback to the person. The prescriptive feedback is generated by one or more feedback devices and indicates a biomechanical change to be made by the person when performing the activity again.

In aspects of the present invention, a system comprises a plurality of sensors attachable to the body of a person performing an activity that includes biomechanical motions, one or more feedback devices, a processor device communicatively coupled to the plurality of sensors and to the one or more feedback devices. The processor device is configured to analyze data from the sensors and configured to send signals to the one or more feedback devices. The one or more feedback sensors provides prescriptive feedback to the person based on the signals sent by the processor device, and the prescriptive feedback indicates a biomechanical change to be made by the person when performing the activity again.

In aspects of the present invention, a non-transitory computer readable medium has a stored computer program embodying instructions, which when executed by a computer system, causes the computer system to provide prescriptive feedback. The computer readable medium comprises instructions for receiving data from on-body sensors attached to the body of a person performing an activity that includes biomechanical motions, instructions for analyzing the data received from the on-body sensors, instructions for providing prescriptive feedback to the person. The prescriptive feedback is provided by one or more feedback devices and indicates a biomechanical change to be made by the person when performing the activity again.

The features and advantages of the invention will be more readily understood from the following detailed description which should be read in conjunction with the accompanying drawings.

### BRIEF DESCRIPTION OF THE DRAWINGS

FIG. 1 is a schematic diagram showing an exemplary system for providing prescriptive feedback on performing a physical activity.

FIG. 2 is a flow diagram showing an exemplary method for providing prescriptive feedback on performing a physical activity.

FIG. 3 is a flow diagram showing an exemplary process for analyzing data from on-body sensors.

FIG. 4 is a chart showing an exemplary stream of data from on-body sensors, the data containing information on biomechanical events and biomechanical motions of a physical activity.

FIG. 5 is a table showing an exemplary prioritization scheme in a process for analyzing data from on-body sensors.

FIG. 6 is a photographic illustration showing an exemplary system for providing prescriptive feedback on performing a basketball shot.

FIG. 7 is a schematic diagram showing an exemplary feedback device in a system for providing prescriptive feedback on performing a physical activity.

FIG. 8 is a schematic diagram showing an exemplary processor device in a system for providing prescriptive feedback on performing a physical activity.

### INCORPORATION BY REFERENCE

All publications and patent applications mentioned in the present specification are herein incorporated by reference to the same extent as if each individual publication or patent application was specifically and individually indicated to be incorporated by reference. To the extent there are any inconsistent usages of words and/or phrases between an incorporated publication or patent and the present specification, these words and/or phrases will have a meaning that is consistent with the manner in which they are used in the present specification.

### DETAILED DESCRIPTION OF EXEMPLARY EMBODIMENTS

A key to helping an individual learn and perfect skills requiring physical dexterity is to observe and analyze the individual's biomechanics and provide immediate prescriptive feedback on how the individual should perform the skill differently for more optimal results. As will be described below, aspects of the present invention are capable of providing these and other functions.

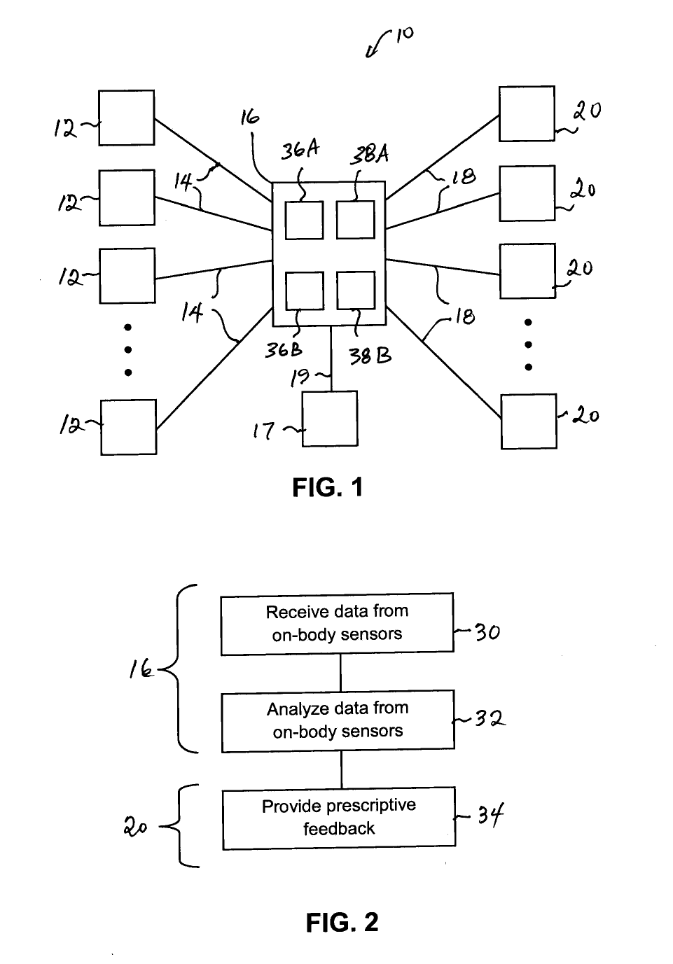

Referring now in more detail to the exemplary drawings for purposes of illustrating exemplary aspects of the invention, wherein like reference numerals designate corresponding or like elements among the several views, there is shown in FIG. 1 exemplary system 10 that generates prescriptive feedback by observing the biomechanical motions of an individual performing an activity. Prescriptive feedback refers to instruction, advice, guidance and the like which is conveyed to the individual for the purpose of guiding the individual on what to do differently so that the individual learns to perform the activity with correct form or in a manner that is desirable.

System 10 analyzes the biomechanical motions relative to an ideal or preferred model of the activity. System 10 identifies and prioritizes what biomechanical motions the individual should do differently to achieve results closer to the model. All this is performed in a timely manner with minimal delay, such as less than 10 seconds, less than 5 seconds, less than 1 second, or less than 0.5 second delay, as the more immediate the feedback, the more effective the feedback will be in guiding the individual to learn and perfect the activity.

Observing the biomechanics of an individual can be performed in a number of ways using on-body sensors 12. Although four on-body sensors 12 are illustrated, system 10 may include a lesser or greater number of on-body sensors 12. There are many types of on-body sensors that can be utilized by system 10, and the various types can be implemented in many different combinations depending on need. What is missing from conventional training systems, however, is a means for analyzing data from the sensors that enables immediate prescriptive feedback to the individual.

The term “on-body” means that the device, such as on-body sensor 12, is attached to the person's body. The device can be attached in direct contact with the skin, or the device can be attached to a garment, strap, shoe, glove, padding, or other item which is worn on and/or secured to the person's body. The way in which the device is attached to the person's body will depend upon the type of device and its capabilities.

On-body sensors 12 can include a combination of motion sensors, myography sensors, and biometric sensors. Motion sensors detect motion in space. Myography sensors detect muscle activity and possibly muscle fatigue. Biometric sensors detect biometric vital signs, including without limitation one or a combination of cardiac activity (e.g., pulse rate and/or variability in pulse rate), respiration rate, blood pressure, and blood chemistry (e.g., oxygen level). There is a wide range of biometrics that can help better understand the biomechanics of an individual. Data from biometric sensors can help correlate changes in biometrics (e.g., changes in biometric vital signs) in response to fatigue or other physical states of the individual. On-body sensors 12 communicate data 14 representative of motion, muscle activity or fatigue, or biometrics depending on the type of on-body sensor.

One or more on-body sensors 12 can be motion sensors, while other on-body sensors 12 can be myography sensors, and other on-body sensors 12 can be biometric sensors. The motion sensors, myography sensors, and biometric sensors can be attached to the same or different locations on the person's body. Locations for attachment will depend on the type of activity (e.g., basketball practice or rehabilitation therapy) and the type of biomechanical motion being monitored (e.g., elbow movement versus knee movement).

As a further example, two or more on-body sensor types (e.g., a motion sensor, myography sensor, and biometric sensor) can be housed in a single on-body sensor 12 that is attached to one location of the person's body, while other on-body sensors 12 constructed in the same way are attached to other parts of the body.

System 10 may include and operate with (1) only motion sensors without myography sensors, (2) only myography sensors without motion sensors, or (3) a combination of motion sensors and myography sensors. For each of the three foregoing variations, system 10 may optionally include biometric sensors.

System 10 includes processor device 16 to which each on-body sensor 12 is communicatively coupled. As used herein, “communicatively coupled” means coupled in a way that enables transmission and/or receipt of data. For example and without limitation, devices that are communicatively coupled to each other can be configured to communicate with each other wirelessly through the air (e.g., radio signals, ultrasonic signals, or optical signals) or through electrical or optical cables. Processor device 16 receives data 14 from each on-body sensor 12. The data is representative of motion, muscle activity or fatigue, or biometrics depending on the type of on-body sensor. Processor device 16 is programmed and/or configured to analyze data 14 and generate signals 18 for prescriptive feedback to be given to the individual on which on-body sensors 12 are attached.

Processor device 16 is communicatively coupled to feedback devices 20 that receive signals 18 generated by processor device 16. Although four feedback devices 20 are illustrated, system 10 may include a lesser or greater number of feedback devices 20. There can be different types of feedback devices 20, including without limitation audible feedback devices, visual feedback devices, and haptic or tactile feedback devices. System 10 can include any one or more of these types of feedback devices. An audible feedback device emits an audible sound, such as a tone or voice message, that guides the individual to perform the activity correctly. A visual feedback device emits a light, such as illuminated arrow or change in color, that guides the individual to perform the activity correctly. A tactile feedback device produces a physical disturbance, such as a mechanical vibration or mechanical pulse, that guides the individual to perform the activity correctly.

Some feedback devices 20 can be on-body feedback devices 20. The definition of the term “on-body” is the same that for on-body sensors 12. Having feedback devices 20 located on the person's body can be beneficial in that it can allow system 10 to communicate in a manner that is intuitive for the individual to understand. For example, a flashing light located on the forearm can indicate a need to change motion of the forearm. A flashing red light at a first part of the body together with a solid green light at a second part of the body can indicate poor biomechanical motion at the first part of the body and good biomechanical motion at the second part. An audible sound emanating from the shoulder and/or a physical disturbance applied to the shoulder can allow the individual to be immediately informed of the area in need of change without the individual having to look at that area of the body. Thus, the individual can be alerted to a problem area while the activity is being performed and/or after the activity has been completed.

One or more feedback devices 20 can be audible feedback devices, while other feedback devices 20 can be visual feedback devices, and other feedback devices 20 can be tactile feedback devices. Each of the feedback devices can be attached to different parts of the body or on the same parts of the body. Locations for attachment will depend on the type of activity and biomechanical motion being monitored.

As a further example, two or more feedback device types (e.g., audible feedback device, visual feedback device, and tactile feedback device) can be housed in a single feedback device 20 attached to one location of the person's body, while other feedback devices 20 constructed in the same way are attached to other locations on the body.

Also, feedback device 20 and on-body sensor 12 can be housed together in a single device, referred to as a sensor node, attached to one location of the person's body while other sensor nodes constructed in the same ware are attached to other locations on the body. Each sensor node is communicatively coupled to processor device 16. Processor device 16 receives data 14 from each sensor node. Each sensor node receives signals 18 from processor device 16.

Referring again to FIG. 1, processor device 16 can infer the current position and rate of motion of an individual's joints from data 14 from on-body sensors 12 (particularly motion sensors) placed on one or more locations on the body. Accurate information can be obtained by using multiple motion sensors located before and after a joint, i.e., on opposite sides of the joint. For example processor device 16 can accurately determine the current biomechanical state of an individual's elbow when on-body sensor 12 is attached to and is used to track orientation in space of the forearm while another on-body sensor 12 is attached to and is used to track orientation in space of the upper arm. Technology to ensure correct relative calibration of motion sensors is described in U.S. Patent Application Publication No. 2014/0150521, entitled “System and Method for Calibrating Inertial Measurement Units.”

With only on-body sensors 12 attached to and used to track motion at only one side of a joint, processor device 16 can infer some information about the joint but it will not be as accurate without motion data from the other side of the joint. For example, with on-body sensor 12 placed only on the upper arm, processor device 16 might infer from data 14 that the arm is raised above the person's shoulder when the sensor detects that the upper arm is pointing up. But, the arm could in fact be at the individual's side and the individual just happens to be turned upside down.

As previously mentioned, on-body sensors 12 can include myography sensors. Processor device 16 can detect which muscles are being used by an individual (and optionally the level of muscle fatigue) from data 14 communicated by myography sensors. Detecting muscle activity can be important for observing biomechanics. In many cases, there are multiple ways an individual can perform an activity in a very similar fashion but using different muscle groups. One type of activity can be performed using one group of muscles, and a very similar but different type of activity (or the same activity performed with poor form) can be performed using another group of muscles. It may desirable to ensure that a specific muscle group is or is not used in the activity. A first muscle group could be preferable over a second muscle group because the first muscle group may allow an individual to perform the activity with greater strength and/or a reduced risk of injury. Detecting muscle use may also be useful when the activity or exercise is being performed to help strengthen a particular muscle group, such as for physical therapy after an injury.

Referring to FIG. 2, at block 30 processor device 16 receives data 14 from on-body sensors 12 while the individual is performing an activity. At block 32, processor device 16 analyzes data 14 to generate signals 18 based on the analysis. Analysis can be performed while the activity is in progress or after the activity has been completed. For example, the activity can include various biomechanical motions performed in sequence. Processor device 16 can analyze each biomechanical motion before other biomechanical motions are performed. At block 34, feedback devices 20 provide prescriptive feedback to the person according to signals 18 received from processor device 16. Prescriptive feedback can be provided while the activity is in progress or after the activity has been completed. For example, feedback devices 20 can provide prescriptive feedback regarding one biomechanical motion for a particular activity before other biomechanical motions for that activity are completed.

In aspects of the invention, analysis (block 32) and/or providing prescriptive feedback (block 34) is performed by system 10 in real-time. As used herein, the term “real-time” means that the function (e.g., analysis and/or providing prescriptive feedback) is completed in less than a second, and optionally less than a tenth of a second, after the occurrence of a biomechanical event.

In aspects of the invention, analysis (block 32) and/or providing prescriptive feedback (block 34) is performed by system 10 in near real-time. As used herein, the term “near real-time” means that the function (e.g., analysis and/or providing prescriptive feedback) is completed within a 1 to 5 second time frame after the occurrence of a biomechanical event.

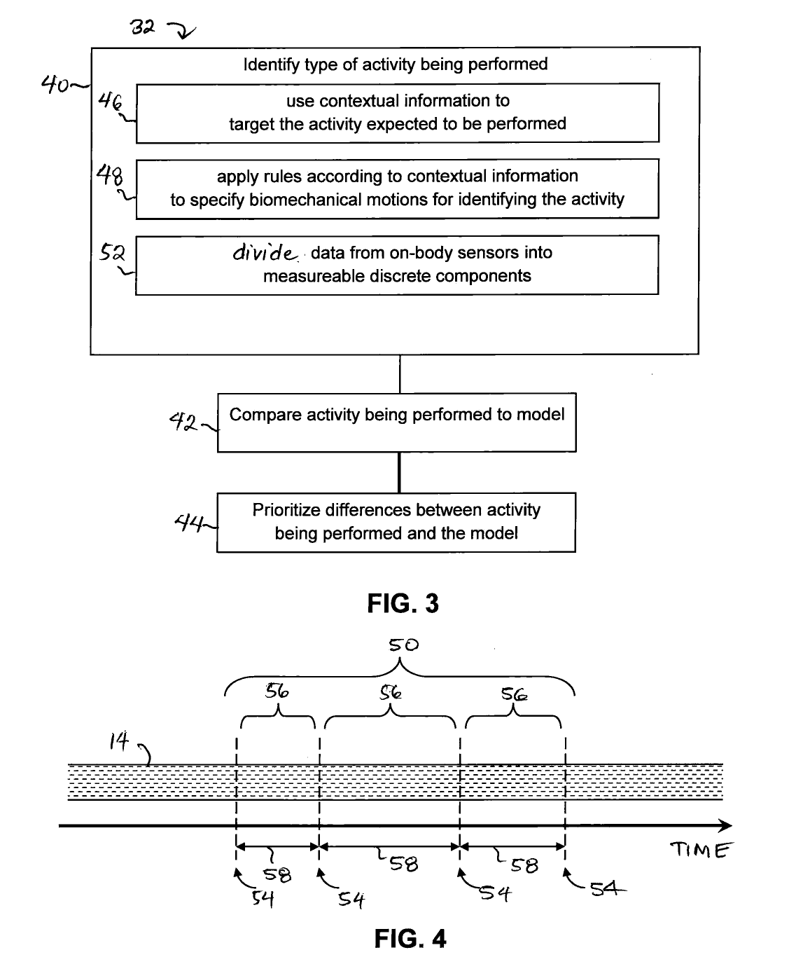

FIG. 3 shows exemplary details of analysis according to block 32 in FIG. 2. At block 40, processor device 16 identifies the type of activity the individual is attempting to perform. Examples of types of activities include without limitation performing a basketball shot, a golf swing, a tennis serve, and a baseball pitch. For rehabilitation therapy after an injury or surgery, the type of activity may include simple tasks such as walking, standing up, sitting down, or even simpler activities that use only a few muscle groups. Each type of activity corresponds to a unique set of biomechanical motions performed in a particular way or sequential order. For example, basketball shots, golf swings, and tennis serves may all involve raising an arm above the shoulder but can each uniquely defined by a different combination of joint angle movements, speed at which the movements are performed, sequence in which the movements are performed, muscles which are activated, type of muscle activation (e.g., isometric contraction versus concentric contraction), and other parameters determined via on-body sensors 12.

At block 42, processor device 16 compares the identified activity to an ideal model of the activity. On-body sensors 12 can be used to create the model of the activity for the individual in advance, before the comparison or any other part of the analysis is performed. The model can include preferred values for the joint angle movements, movement speed, time sequence of movements, muscle activation, and/or other parameters. The comparison includes identifying differences between: (a) the biomechanical motions in the activity performed by the individual, and (b) the preferred values as specified in the model. At block 44, processor device 16 prioritizes the differences. Prioritization involves determining what information will be communicated to the individual as part of the prescriptive feedback and what information will not be communicated to the individual.

Identifying what motion an individual is attempting to perform is in general a difficult problem, but in many cases there is contextual information about what the individual is doing. Processor device 16 can be instructed to detect a specific activity which allows processor device 16 to rapidly identify with high confidence the specific activity when it is performed. For example, the activity of performing a basketball shot can be specifically targeted by system 10 when the individual is known to be practicing basketball shots. When practicing, the individual will be performing various other motions involved in preparing for the basketball shot, retrieving the basketball in order to perform additional basketball shots, and/or passing the ball to another person who is also practicing basketballs shots. Processor device 16 can be set by the individual or a coach to monitor only those biomechanical motions that are specific to a basketball shot. Biomechanical motions specific to a basketball shot can be specified by monitoring rules stored in or provided to processor device 16. Such monitoring rules may indicate that the combination of (a) rapidly raising an arm in the air and (b) stopping at a particular range of angles for the elbow and wrist means that the individual is performing a basketball shot. Thus, each time the individual's arm is raised rapidly and stops within the specified range of angles for the elbow and wrist, as detected from data 14 from on-body sensors 12, processor device 16 can infer that the individual is most likely performing a basketball shot and not preparing for the shot, retrieving the basketball, or passing the basketball to another person. Similarly for other sports and various therapy situations, a set of monitoring rules applied according to the contextual situation can be implemented to enable processor device 16 to rapidly identify a targeted activity with high reliability.

As indicated at block 46 in FIG. 3, identification of the type of activity the individual is attempting to perform optionally includes using contextual information to set processor device 16 to monitor one particular activity or a reduced number activities. For example, processor device 16 may be capable of identifying many different activities involved in a particular sport or a variety of sports, but the individual may be practicing only one or two of those activities. Thus, the individual can activate user input device 17 (FIG. 1), such as keypad or touch screen communicatively coupled to processor device 16, to generate signal 19 indicative of the contextual situation. For example, the individual may press a button or make a menu selection on graphical user interface to indicate what activity should be targeted by processor device 16. Signal 19 is received by processor device 16 so that processor device 16 will attempt to identify only the particular activities that the individual will actually be performing. As a result, processor device 16 can disregard all other types of activities it is capable of identifying and thereby apply more computing resources to the identification and analysis of the activities that the individual will be practicing. After contextual information 19 is provided by the individual to processor device 16, processor device 16 gains access to and applies monitoring rules (block 48 in FIG. 3) that specify the particular biomechanical motions and events that are useful for identifying the particular activities that the individual will be performing.

For example, when contextual information 19 specifies that a basketball shot is the targeted activity, processor device 16 can apply a first set of monitoring rules 36A (FIG. 1) corresponding to a basketball shot. Monitoring rules 36A may specify that a basketball shot is to be identified by the occurrence of an arm being raised rapidly and stopping so that the arm is oriented with a particular range of angles for the elbow and wrist. When contextual information specifies that a tennis serve is the targeted activity, processor device 16 applies a second set of monitoring rules 36B corresponding to a tennis serve. Second set of monitoring rules 36B may specify that a tennis serve is to be identified by the occurrence of an arm being raised rapidly and stopping so that the arm is oriented with a particular range of angles for the elbow and wrist that are different from those for a basketball shot. Processor device 16 will monitor data 14 to detect the particular biomechanical motions specified by monitoring rules 36B and may disregard the biomechanical motions specified by monitoring rules 36A. Although two sets of monitoring rules 36A, 36B are depicted, system 10 can have only a single set of monitoring rules or more than two sets of monitoring rules. The number of monitoring rules may depend on the type of sport or rehabilitation therapy and may also depend on the needs of an athlete or patient.

As indicated at block 52 in FIG. 3, monitoring of data 14 for targeted activity 50 may include dividing data 14 into a set of measurable discreet components.

In FIG. 4, the horizontal line represents time, and the bar schematically represents the stream of data 14 from all on-body sensors 12 to processor device 16. An aspect of dividing activity 50 into measurable discreet components is to identify particular biomechanical events relevant to the activity. Biomechanical events are detectable points 54 that are expected to occur during the activity. Detectable points 54 include the start of a biomechanical motion and a momentary pause in movement between the end of one biomechanical motion and the start of another biomechanical motion. For example, for a golf drive a suitable biomechanical events can include the moment an individual starts a backswing for a golf drive, the moment the individual reaches the back of the backswing and comes to a momentary stop before proceeding to their forward swing, and the moment the individual starts to break (i.e., flex) his wrists during the swing. Each of these biomechanical events 54 can be identified by processor device 16, through analysis of data 14 from on-body sensors 12, to determine when the individual is performing a golf drive. At any one or more of biomechanical events 54, data 14 will provide measurements of the biomechanical motion (e.g., orientation and/or angle of the individual's limbs) that can be used by processor device 16 to determine when the individual is performing the targeted activity. The type and number of detectable points 54 utilized by processor device 16 will depend on the targeted activity.

Biomechanical events 54 (also referred to as detectable points 54) include without limitation the person entering or attaining a particular body position (e.g., the person attaining a body orientation corresponding to a set position prior to throwing a basketball or corresponding to completion of a backswing for a golf drive), departing or moving out of a particular body position, initiating a biomechanical motion (e.g., starting a forward swing or backswing of the golf club, or starting to throw the basketball), completing a biomechanical motion, and pausing in the midst of a biomechanical motion.

As indicated above, targeted activity 50 may comprise multiple biomechanical motions 56 (FIG. 4) which are performed in a particular sequence, and monitoring data 14 for targeted activity 50 can involve dividing data 14 into measureable discrete components. The measureable discrete components can be parts of data 14 at detectable points 54 (e.g., start of biomechanical motion, and a momentary pause in movement between the end of one biomechanical motion and the start of another biomechanical motion). Also, the measureable discrete components can be the timing of all the biomechanical events (e.g., the sequential order in which detectable points 54 occur and/or the amount of time between detectable points 54) and relevant biomechanical measurements (e.g., angle and rate of motion) at or between detectable points 54.

Biomechanical events are the detectable points which are expected to occur during the targeted activity. The processor device 16 determines from data 14 the sequential order in which biomechanical events 54 occurred and the amount of time 58 (FIG. 4) between biomechanical events 54. At each biomechanical event 54, processor device 16 extracts from data 14 measurements of angles and rates of motion of body parts (either an absolute measurement or a relative measurement compared to another body part). In addition or alternatively, processor device 16 may extract from data 14 measurements of muscle activation and exertion and optionally biometrics like heart rate or phase and rate of respiration. Measurements can be taken once during biomechanical event 54. Also, multiple measurements between biomechanical events 54 can be aggregated, such by averaging or determining maximum or minimum values. The measurement, either a single measurement or an aggregate of measurements, is then examined by processor device 16 to determine whether the targeted activity was likely to have been performed by the individual. The type of measurement (e.g., rate of motion, timing, etc.) and the nature of measurements (e.g., single measurement, or aggregated measurement) which are appropriate can depend on the type of activity being targeted, can be predetermined based on expertise and experience related to the targeted activity, and can be defined by monitoring rules 36A, 36B (FIG. 1) accessed by processor device 16.

As previously mentioned, processor device 16 compares the detected activity to a model (block 42 in FIG. 3). The model represents an ideal or preferred way of performing the activity. For a targeted activity, the types of measurable discrete components of data 14 (e.g., the angle of particular joints, the sequence of biomechanical events, the time separating biomechanical events, etc.) and corresponding requirements (e.g., angle values, speed values, sequential order, time values, etc.) are known in advance prior to performing the analysis. For example, the types of measurable discrete components of data 14 which processor device 16 should look for, can be specified in monitoring rules 36A, 36B used by processor device 16 to identify when the targeted activity is being performed.

Requirements for the discrete components of data 14 are defined by the model and can be stored in a data base, lookup table, or algorithm which is accessed or implemented by processor device 16. There can be different models 38A, 38B (FIG. 1), with each model being unique to a particular activity. Although two models 38A, 38B are depicted, system 10 can have only a single model or more than two models. The number of models may depend on the type of sport or rehabilitation therapy and may also depend on the needs of an athlete or patient.

Each model 38A, 38B can be derived in advance from expert human knowledge of what “correct” form should be. An expert may use her knowledge of biomechanical rules for performing the activity with correct form. For example, one biomechanical rule for correct form may dictate that an individual's non-dominate arm should not bend during the back swing and first half of the forward swing of a golf drive. The biomechanical rules for correct form may be derived from common knowledge of persons familiar with the activity.

Additionally or alternatively, the requirements for the discrete components of data 14 defined by the model can be extracted from an expert performing the targeted activity. Sensors, such as on-body sensors 12 or similar technology, can be attached to the expert's body to determine the requirements as well as what biomechanical events 54 are particularly relevant and useful for detecting the targeted activity. Once an expert performs the activity “correctly,” measurements of the set of biomechanical motions performed by the expert can be used as the model which the individual will be guided to emulate through prescriptive feedback from feedback devices 20.

While the use of biomechanical rules and measurements taken from the body of an expert are useful for constructing a model of an activity, especially for an individual trying to learn a new skill, these approaches have limitations. Each individual has slightly different biomechanics, different lengths of body segments, different degrees of freedom in joints and different muscle strength. So the “correct” form for each individual will be slightly different, especially when one looks at very small differences. To address this person-to-person variation, the model can be developed by having the individual perform the action once or a few times while on-body sensors 12 are attached to the individual in order to record the biomechanical motions corresponding to “correct” form. This personal calibration process can be performed by an individual who is already skilled in performing the targeted activity but simply wants to model his motion so he can improve consistency of performance. For a novice, the personal calibration process can be performed with the assistance of an expert who works closely with the novice so that the novice can perform the activity correctly. A coach could work with a player to get her to perform a swing or shot correctly, and the coach can identify that action which was the player's “personal best.” Data from on-body sensors 12 recorded during the personal best performance can be used to construct the model.

As a further example, a physical therapist can work with a patient, even physically manipulating the body of the patient, to perform an exercise correctly while recording data from on-body sensors 12 for use in constructing a model of an activity. During the personal calibration process, processor device 16 uses data 14 to construct a model against which subsequent performance of the activity will be compared. Thus, the model is personalized to an individual's unique biomechanics. The system then helps the individual learn to consistently repeat the activity with minimal variation from a model based on the previously recorded activity.

In other aspects, the model can be constructed through a combination of the previously described approaches. For example, on-body sensors 12 and processor device 16 can be used to record an individual's best attempt in performing an activity to provide initial requirements for biomechanical motions. Thereafter, the initial requirements are modified to develop final requirements which are then used during subsequent analysis (e.g., block 32 in FIG. 2). The modification of initial requirements can be based on expert knowledge of biomechanical rules for correct form and/or data collected from on-body sensors 12 attached to the expert's body while the expert is performing the activity. The modification of initial requirements can be accomplished interactively, such as through the use of a graphical user interface of input device 17 (FIG. 1), to allow an individual or an expert to refine individual parameters of the model.

As mentioned above, system 10 can prioritize what feedback is provided to the individual regarding the activity performed by the individual. After the activity is divided into measureable discrete components, as previously described above, requirements for the measureable discrete components are compared to a model. Thousands or millions of data points may be analyzed and divided into tens of hundreds of measurements. Depending on the complexity of the activity, processor 12 may detect a multitude of differences between the measurements and requirements. System 10 can take all that information and apply a prioritization process that generates simplified feedback to the individual while the action is being performed (e.g., feedback about a biomechanical motion is provided at the start or middle of the activity) or after performance of the activity has been completed.

Prioritization can be beneficial in that the individual can be provided with guidance on a few facets of the activity so the individual is not overwhelmed with too much information. The prioritization process can utilize the degree by which a biomechanical motion diverges from the model and utilize a preset ranking of measurements. The ranking of measurements can be based upon what experts have determined to be the most and least important measurements for the targeted activity.

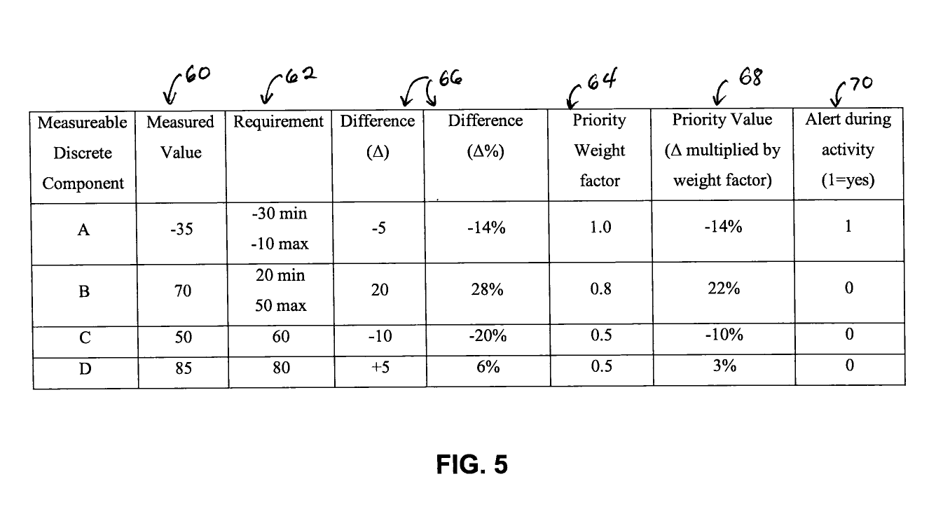

Referring to FIG. 5, data 14 includes measurements for various measurable discrete components A-D (referred to as metrics for convenience) for an activity performed by the individual. For example, metric A could represent the angle of the hip at the start of a biomechanical motion, metric B could represent acceleration of the upper leg during the biomechanical motion, metric C could be the angle of the hip at the end of the biomechanical motion, and metric D could be the acceleration of the foot during a subsequent biomechanical motion. Processor 12 extracts measurements 60 for the metrics from data 14 and compares measurements 60 to requirements 62 defined in the model. The metrics can represent other biomechanical parameters, such as those associated with an arm or other body part. Although four metrics are depicted, there can be a lesser or greater number of metrics. The appropriate number of metrics can depend on the type of targeted activity.

The prioritization process looks at both how far each metric is from its requirement as well as a priority scheme for the metrics. The priority scheme embedded within system 10 can be based on knowledge of an expert on what to look for and focus on and codifies that knowledge for use by processor device 16. Metrics that are considered by the expert to be more important can be given a greater weighting factor 64 than metrics considered less important. Along with weighting factor 64 is the amount 66 by which measurements of actual motion differ from requirements 62. Processor device 16 combines priority weighting 64 and differences 66 to determine priority values 68 which are then used to determine the contents of prescriptive feedback. This can be performed, for example, by multiplying priority weight 64 by measured difference 66 and then selecting metrics which the highest priority value 68. Processor device 16 may generate signals 18 which notify the individual to make changes in his biomechanical motion according to the two metrics (e.g., metrics A and B in FIG. 5) with the highest absolute priority value 68. In other aspects, signals 18 are generated only for a single metric (e.g., metric B) with the highest absolute priority value 68 or the metrics (e.g., metrics A, B, and C) with the top three absolute priority values 68.

Other weighting methods or prioritization schemes can be implemented. For example, it may be important for the individual to be alerted of a need to change his biomechanics while the activity is being performed. Importance may be due to a need to avoid injury. An alert signal from feedback device 20 can reduce the risk of injury by notifying the individual to stop a motion that he is performing in a dangerous manner. In some cases providing feedback during the action can help guide the individual better to understand what he needs to do differently. Thus, the prioritization scheme may include flags 70 that instruct processor device 16 to generate signal 18 during the activity (before the activity is complete) for some of the metrics (metric A in FIG. 5) but not for other metrics.

In further aspects, different priority weights can be applied depending on whether the measurement of the metric is above or below the requirement. For example, a greater priority weight can be applied when measurement 60 is greater than requirement 62 (e.g., when difference 66 is positive), possibly because there is a greater risk of injury as compared to when measurement 60 is less than requirement 62 (e.g., when difference 66 is negative).

In further aspects, processor device 16 may wait until the activity is complete, and then begin the prioritization process to determine what feedback will be given to the individual.

In further aspects, processor device 16 can divide the metrics into different groups and then select only the metric with the highest priority value 68 in each group. For example, metrics A and B can be related to one biomechanical motion so they are considered to be a first group, and metrics C and D can be related to a subsequent biomechanical motion so they are considered to be a second group. Based on priority values 68 in FIG. 5, processor device 16 may send signals 18 to feedback devices 20 to guide the individual to make changes to his biomechanical motion according to measurements for metrics B and C only. Although the absolute priority value of metric A is greater than that for metric C, processor device 16 does not send signals 18 to feedback devices 20 regarding metric A. Thus, due to the way in which metrics were grouped together, the prescriptive feedback given to the individual is based exclusively on metrics B and C.

Various other criteria can be implemented for determine which metrics belong in the same group. For example, one group may consist of metrics for angles of joints, and another group may consist of metrics for acceleration of various limbs. As a further example, one group may consist of metrics for one area of the individual's body, and another group may consist of metrics for a different area of the body.

For the feedback to be useful, it should be prescriptive in the sense that the individual is notified of what to do differently to improve performance of the targeted activity. To provide prescriptive feedback, system 10 can look at which measurement was prioritized and in what direction the difference is (e.g., measurement is greater than or less than the requirement) and then provides feedback on what to do differently. This can involve taking knowledge from an expert which is codified in the prioritization process previously discussed.

The prescriptive feedback in many different forms, such as audio, visual, and haptic, can be given by feedback devices 20 to the individual. An exemplary form of audio feedback is playing tones to indicate that the user made a mistake. A sequence of tones can be used to identify what the mistake was. Another form of audio feedback is to provide verbal feedback to tell the individual what to do differently. This can be performed by processor device 16 by transmitting signals 18 for prerecorded voice sequences. Also, this can be performed by processor device 16 by computing synthesized text to speech. Thus, feedback device 20 can provide verbal commands that tell the individual what to do differently in his biomechanics to perform the action closer to the model.

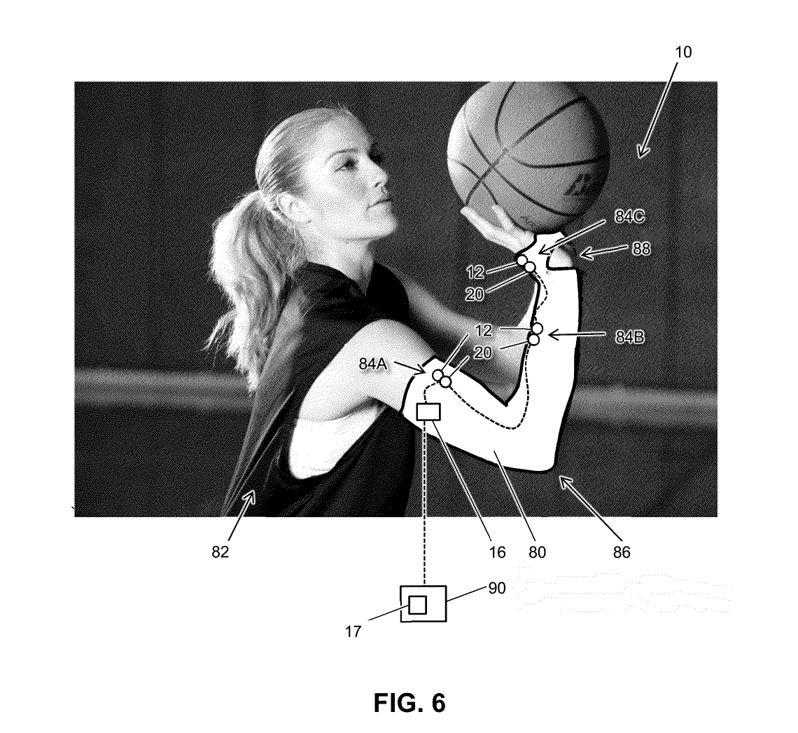

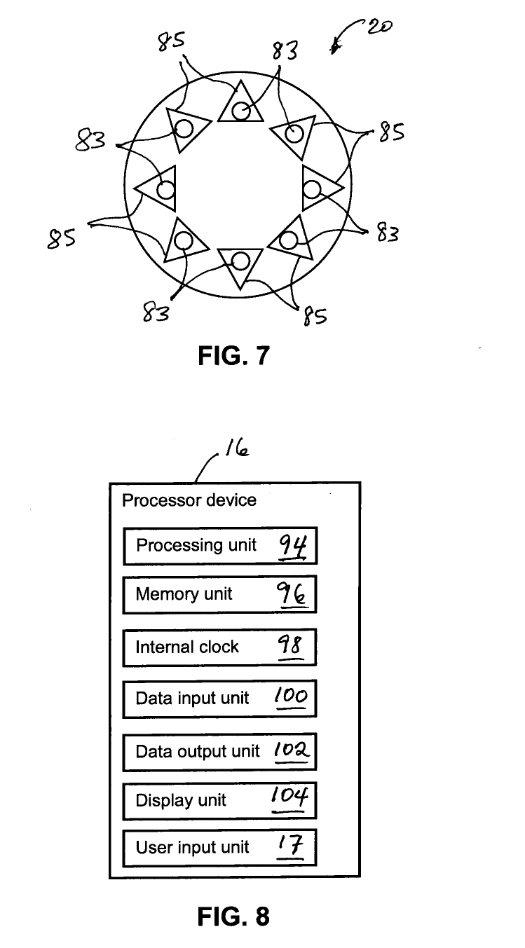

Visual feedback can be in the form of a graphic representation of what the individual should do differently, for example through animation on display unit 104 (FIG. 7) of processor device 16 or the display screen of mobile device 90 (FIG. 6). Thus, the display screen of processor device 16 or mobile device 90 can serve as feedback device 20. Another form of visual feedback can be lighting up arrows directly on the body. The arrows can indicate how the individual should move their body, as will be discussed in connection with FIG. 7.

Additionally or alternatively, prescriptive feedback can be haptic or tactile feedback. The words “haptic” and “tactile” are used interchangeably herein. Small mechanical within feedback device 20 attached to different points on the body can be used to indicate how the individual performed the action or how the individual should perform the action. The direct physical correspondence of the actuator placement on the body to what body part should move differently provides an intuitive feedback. The haptic feedback can be performed to draw attention to a body part that moved incorrectly. By placing actuators on opposing sides of a garment, such as a sleeve or legging, one can indicate the direction in which the individual should move a particular body part to perform the action closer to the model. Haptic feedback can be very useful for feedback while an individual is performing the action to help guide them. System 10 can provide immediate, direct haptic feedback when biomechanical motion diverges from the model.

It is important for prescriptive feedback to be conveyed by feedback devices 20 to the individual in an effective manner. One of the more effective forms of feedback is on-body prescriptive visual feedback provided by on-body visual feedback devices 20 mentioned previously. On-body visual feedback devices 20 may produce a visual indicator comprising changes in color or illumination on the individual's body to convey what the individual should do differently to achieve the better results. The visual indicators can be produced by light emitting diodes (LEDs), lamps, or other light sources. The light sources can be flexible, such as a flexible strip or flexible optical fiber, so that they can be incorporated into or otherwise attached to a garment, fabric or other article that is secured to the individual. The visual indicators can make a selected portion of the garment (fabric or other article) appear to change color. The visual indicators can be positioned on the body such that the location of the indicator corresponds to body parts, muscle groups or specific joints which are being monitored by on-body sensors 12.

Visual indicators produced by on-body visual feedback devices 12 can provide feedback to the individual as to what the individual just did biomechanically and optionally point out what the individual may have done correctly or incorrectly in performing an action. For example one can use a set of LEDs to illuminate an arrow to indicate the direction in which the individual should move a body part to perform the action closer to the model.

In FIG. 6, system 10 includes on-body sensors 12 that are mounted on fabric sleeve 80 which can be worn while playing a sport such as basketball. System 10, which is in the form of a training sleeve, can provide a basketball player with feedback on jump shots and free throws.

On-body sensors 12 attached to fabric sleeve 80 detect the primary shooting arm of athlete 82. Sleeve 80 mounts on-body sensors 12 to the arm of athlete 82. On-body sensors 12 enable processor device 16 to detect when athlete 82 attempts a basketball shot (as opposed to another maneuver, such as dribbling the basketball ball) and to analyze the form of the basketball shot. Athlete 82 can receive immediate feedback through audio and visual indicators produced by on-body feedback devices 20 coupled to on-body sensors 12.

On-body feedback devices 20 can include lights (e.g., light emitting diodes or lamps) and/or speakers or other device configured to generate a sound. When the athlete's form is incorrect or undesirable, on-body feedback devices 20 emit a light and/or sound to indicate how to improve future basketball shot. Athlete 82 may also track her performance and compare it to that of teammates using a software application program running on mobile device 90 communicatively coupled to processor device 16. Examples for mobile device 90 include without limitation a smartphone, tablet computer, and laptop computer. Mobile device 90 can be owned or operated by athlete 82 or another person.

Training sleeve 10 includes three on-body sensors 12: one on the back of the hand, one on the forearm, and one on the upper arm. Each on-body sensor 12 is a motion sensor that comprises a 3-axis accelerometer, a 3-axis gyroscope, and a 3-axis compass which, in combination, accurately track rotation and motion in space using algorithms. On-body sensors 12 are communicatively coupled to processor device 16 which applies the algorithms to sensor data 14. On-body sensors 12 are sampled by processor device 16 at around 200 times per second. From sensor data 14, processor device 16 can determine the current rotation of the shoulder, elbow, and wrist.

Optionally, on-body feedback devices 20 and on-body sensors 12 are housed together in various sensor nodes 84A-C. Each sensor node is located on a different part of the arm. Sensor nodes 84A and 84B are located on opposite sides of elbow joint 86. Sensor nodes 84B and 84C are located on opposite sides of wrist joint 88. This arrangement allows processor device 16 to determine the angles of the elbow and wrist during various biomechanical events (e.g., start of biomechanical motion, and a momentary pause in movement between the end of one biomechanical motion and the start of another biomechanical motion) and during various biomechanical motions. Also, this arrangement allows processor device 16 to measure the rate of rotational movement of the upper arm via sensor node 84A, forearm via sensor node 84B, and wrist via sensor node 84C.

As shown in FIG. 7, feedback device 20 can have a ring of eight light sources 83. With the ring of light sources (such as LEDs or lamps), training sleeve 10 can indicate with arrows 85 the direction in which a body part (e.g., upper arm, forearm, or wrist) should be moved or should have been moved to perform a correct action. Each light source 83 and corresponding arrow 85 together represent a different direction. For example, when athlete 82 shoots a basketball shot with her elbow too far out, one light source 83 on the forearm may illuminate arrow 85 that points inward toward the athlete's body to prompt the athlete to keep her arm closer to the body. The arrow can be illuminated while the athlete is shooting the basketball whenever the arm goes out too far away from the body. Alternatively, the arrow can be illuminated after completion of the basketball shot.

Although eight light sources 83 are depicted, each feedback device 20 may have a lesser or greater number of light sources to indicate direction. The appropriate number of light sources may depend upon the activity being performed and the body part on which feedback device 20 is attached.

Processor device 16 uses sensor data 14 from on-body sensors 12 to detect when the athlete performs a basketball shot and analyzes whether the action was performed with good or bad form. The detection of a basketball shot and analysis are performed using algorithms running in processor device 16. The basketball shot is broken down into many measurable discrete components (such as metrics A-D in FIG. 5). Measurements for the discrete components can include without limitation joint angles, acceleration, rotation, and direction of movement. For each measurable discrete component, the requirement for good form is defined by a model. The requirements contained in the model can be configured or modified by athlete 82 or other person using the software application program running on mobile device 90 and input device 17 (such as a touch sensitive screen or keyboard) of mobile device 90. The software application program allows the model to be tailored to athlete 82.

As indicated above, athlete 82 can get immediate prescriptive feedback through audio and visual indicia from on-body feedback sensors 20. Processor device 16 causes feedback sensors 20 to provide immediate feedback after a basketball shot by either playing a sequence of tones and/or by speaking to the player to provide guidance.

Processor device 16 can communicate with mobile device 90 using Bluetooth or other wireless communication protocol. This can allow all sensor data 14 from training sleeve 10 to be uploaded into a cloud storage environment. A cloud storage environment refers to storage of data in any number of computer servers at any number of physical locations, and the computer servers are owned and managed, not by the individual using training sleeve 10, but by a hosting company. Further analysis as well as tracking of performance over time can be performed either on mobile device 90, in the cloud, or both. Mobile device 90 can also be used to personalize settings for one or more athletes, as well as to update the software and algorithms running on processor device 16.

In any of the aspects described in association with FIGS. 1-7, processor device 16 can include various components as shown in FIG. 8. In FIG. 8, exemplary processor device 16 includes processing unit 94 that analyzes data 14 received from on-body sensors 12. Although processor device 16 is schematically depicted as a single box, it should be understood that various components of processor device 16 can be housed together in a single case or can be housed in separate cases while still being communicatively coupled with each other.

Processing unit 94 can include one or more circuit assemblies, microprocessors and electronic semiconductor chips. Memory unit 96 includes one or more memory components, e.g., components for volatile and/or non-volatile data storage, for storing data 14 received from on-body sensors 12. Internal clock 98 enables processor device 16 to keep track of time between biomechanical events. Data input unit 100 is configured to receive data 14 from on-body sensors 12. Data input unit 100 may include various components (e.g., antennas, electrical connectors, and data processing circuitry) that allow data 14 to be received wirelessly through the air (e.g., via radio signals or other electromagnetic radiation in the air) or by wire (e.g., electrical or fiber optic cable).

Optionally, processor device 16 may also include data output unit 102 that enables processor device 16 to export data to another device, such as mobile device 90 (FIG. 6). Data output unit 102 may include various components (e.g., antennas, electrical connectors, and data processing circuitry) that allow data 14 or results of data analysis to be transmitted wirelessly or by wire. Data output unit 102 may also handle transmission of signals 18 to feedback devices 20. Processor device 16 may also include display unit 104 that enables processor device 16 to visually display text and/or graphics that represent data 14, results of data analysis, and prescriptive feedback. Display unit 104 can be a liquid crystal display screen, light emitting diode display screen, other type of electronic display. Processor device 16 may also include user input unit 17 that allows a person to adjust requirements defined in the model of a targeted activity. Input unit 17 can be a keyboard, touch sensitive screen, microphone, or a remote control button.

Processor device 16 can be capable of executing, in accordance with a computer program stored on a non-transitory computer readable medium, any one or a combination of the steps and functions described above for receiving data 14, analyzing data 14, and providing prescriptive feedback. The non-transitory computer readable medium may comprise instructions for performing any one or a combination of the steps and functions described herein. Processor device 16 (optionally memory unit 96) may include the non-transitory computer readable medium. Examples of a non-transitory computer readable medium includes without limitation non-volatile memory such as read only memory (ROM), programmable read only memory, and erasable read only memory; volatile memory such as random access memory; optical storage devices such as compact discs (CDs) and digital versatile discs (DVDs); and magnetic storage devices such as hard disk drives and floppy disk drives.

In any of the aspects described in association with FIGS. 1-8, on-body sensors 12 can include an inertial measurement unit (IMU), which is a type of motion sensor. The IMU is configured to detect motion of the body. The IMU can be the ones described in U.S. Patent Application Publication No. 2014/0150521 (titled “System and Method for Calibrating Inertial Measurement Units”). An IMU is configured to provide information on its orientation, velocity, and acceleration. An IMU may include gyroscopes, accelerometers, and/or magnetometers. A gyroscope is configured to measure the rate and direction of rotation. An accelerometer is configured to measure linear acceleration. A magnetometer (a type of compass) is configured to detect direction relative to magnetic north pole.

As previously mentioned, on-body sensors 12 can also include myography sensors configured to detect whether a particular muscle is being used by the person and optionally how fatigued that muscle is. Myography sensors include sensors configured to provide signals indicative of muscle contraction, such as signals corresponding to electrical impulses from the muscle, signals corresponding to vibrations from the muscle, and/or signals corresponding to acoustics from the muscle, as described in U.S. Patent Application Publication No. 2014/0163412 (titled “Myography Method and System”). Other exemplary myography sensors include those described in U.S. Patent Application Publication Nos. 2010/0262042 (titled “Acoustic Myography Systems and Methods”), 2010/0268080 (titled “Apparatus and Technique to Inspect Muscle Function”), 2012/0157886 (titled “Mechanomyography Signal Input Device, Human-Machine Operating System and Identification Method Thereof”), 2012/0188158 (titled “Wearable Electromyography-based Human-Computer Interface), 2013/0072811 (titled “Neural Monitoring System”), and 2013/0289434 (titled “Device for Measuring and Analyzing Electromyography Signals”).

Myography sensors include without limitation a receiver device configured to detect energy which has passed through the person's body or reflected from the person's body after having been transmitted by a transmitter device. The receiver device need not be in contact with the person's skin. Myography sensors with these types of receiver and transmitter devices are described in U.S. Patent Application Publication No. 2015/0099972 (titled “Myography Method and System”). The type of energy transmitted by the transmitter device and then received by the receiver device includes without limitation sound energy, electromagnetic energy, or a combination thereof, which are used to infer vibrations occurring on the skin surface, below the skin surface, or in the muscle which naturally arise from muscle contraction. For example, the transmitter device can be configured to transmit (and receiver device can be configured to detect) audio signals, which can include acoustic waves, ultrasonic waves, or both. Acoustic waves are in the range of 20 Hz to 20 kHz and include frequencies audible to humans. Ultrasonic waves have frequencies greater than 20 kHz. Additionally or alternatively, transmitter can be configured to transmit (and receiver 16 can be configured to detect) radio waves. For example, radio waves can have frequencies from 300 GHz to as low as 3 kHz. Additionally or alternatively, the transmitter device can be configured to transmit (and receiver device can be configured to detect) infrared light or other frequencies of light. For example, infrared light can have frequencies in the range of 700 nm to 1 mm. These types of energy, after having passed through the person's body or reflected from the person's body, are analyzed by processor device 12 to infer muscle contraction and/or muscle fatigue.

While several particular forms of the invention have been illustrated and described, it will also be apparent that various modifications can be made without departing from the scope of the invention. It is also contemplated that various combinations or subcombinations of the specific features and aspects of the disclosed embodiments can be combined with or substituted for one another in order to form varying modes of the invention. Accordingly, it is not intended that the invention be limited, except as by the appended claims.

1. A method for generating prescriptive feedback about an activity performed by a person, the method comprising:
receiving data from on-body sensors attached to the body of a person performing an activity that includes biomechanical motions;
analyzing the data received from the on-body sensors; and
providing prescriptive feedback to the person, wherein the prescriptive feedback is generated by one or more feedback devices and indicates a biomechanical change to be made by the person when performing the activity again.

2. The method of claim 1, wherein each on-body sensor includes a motion sensor.

3. The method of claim 1, wherein each on-body sensor is mounted on a garment worn by the person, and the analyzing is performed by a processor device mounted on the garment.

4. The method of claim 1 wherein analyzing includes dividing the data received from the on-body sensors into measureable discrete components, and the measureable discrete components include any one or more of a measurement of an angle of a body part of the person, a measurement of a rate of motion of a body part of the person, and a measurement of time.

5. The method of claim 4, wherein dividing the data includes identifying biomechanical events within the data, the biomechanical events being any one or more of:
attainment of a particular body position, departure from a particular body position, initiation of a biomechanical motion, completion of a biomechanical motion, and a pause in the midst of a biomechanical motion.

6. The method of claim 4, wherein the measureable discrete components include any one or more of: a biomechanical measurement at one of the biomechanical events, and an aggregate of measurements taken between two of the biomechanical events.

7. The method of claim 1, wherein analyzing includes comparing measurements of the biomechanical motions to requirements defined in a model of the activity.

8. The method of claim 7, further comprising modifying the model by changing the requirements against which measurements are to be compared.

9. The method of claim 8, wherein modifying the model is performed by the person performing the activity or another person through the use of a graphical user interface communicatively coupled to a processor device that performs the analyzing.

10. The method of claim 1, wherein the prescriptive feedback specifies a part of the body which the person should move differently.

11. The method of claim 10, wherein the prescriptive feedback specifies a characteristic of the movement that should be changed by the person when the person performs the movement again, and the characteristic includes any one or more of: acceleration of the body part in space, orientation of the body part relative to another body part, rotation of the body part relative to another body part, rate of motion of the body part, and direction of motion of the body part.

12. The method of claim 1, wherein the one or more feedback devices are attached to parts of the person's body that carry out the biomechanical motions of the activity.

13. The method of claim 1, wherein the prescriptive feedback includes a visual indicator that specifies the biomechanical change to be made by the person when performing the activity again.

14. The method of claim 13, wherein the one or more feedback devices includes a light source that illuminates or changes color to produce the visual indicator.

15. The method of claim 13, wherein the visual indicator indicates a direction for the biomechanical change.

16. The method of claim 13, wherein the visual indicator includes a video animation displayed on a display screen communicatively coupled to a processor device that performs the analyzing.

17. The method of claim 1, wherein the prescriptive feedback includes audible tones that specify the biomechanical change to be made by the person when performing the activity again.

18. The method of claim 1, wherein the prescriptive feedback includes a verbal command that specifies the biomechanical change to be made by the person when performing the activity again.

19. (canceled)

20. A system for generating prescriptive feedback about an activity performed by a person, the system comprising:
a plurality of sensors attachable to the body of a person performing an activity that includes biomechanical motions;
one or more feedback devices; and
a processor device communicatively coupled to the plurality of sensors and to the one or more feedback devices, the processor device configured to analyze data from the sensors and configured to send signals to the one or more feedback devices,
wherein the one or more feedback sensors provides prescriptive feedback to the person based on the signals sent by the processor device, and the prescriptive feedback indicates a biomechanical change to be made by the person when performing the activity again.

21-38. (canceled)

39. A non-transitory computer readable medium having a stored computer program embodying instructions, which when executed by a computer system, causes the computer system to provide prescriptive feedback, the computer readable medium comprising:
instructions for receiving data from on-body sensors attached to the body of a person performing an activity that includes biomechanical motions;
instructions for analyzing the data received from the on-body sensors; and
instructions for providing prescriptive feedback to the person, wherein the prescriptive feedback is provided by one or more feedback devices and indicates a biomechanical change to be made by the person when performing the activity again.

40-56. (canceled)
